(19) United States
(12) Patent Application Publication
(10) Pub. No.: US 2024/0024165 A1
(43) Pub. Date: Jan. 25, 2024

(54) TAMPON CONTAINING A PHARMACEUTICAL COMPOSITION

(71) Applicant: Swiss-Canna Group AG, Zürich (CH)

(72) Inventors: Nabila Meier, Hamburg (DE); Patrick Meier, Hamburg (DE)

(73) Assignee: Swiss-Canna Group AG, Zürich (CH)

(21) Appl. No.: 18/037,637

(22) PCT Filed: Nov. 20, 2020
(86) PCT No.: PCT/EP2020/082952

§ 371 Date: May 18, 2023

(51) Int. Cl. A61F 13/20; A61K 31/00; A61K 36/28

(52) U.S. Cl. CPC A61F 13/2074; A61F 13/2085; A61K 31/658; A61K 36/28

(57) ABSTRACT

This disclosure presents and discusses a tampon having an absorbent main part that may be made of cotton. The main part includes a pharmaceutical composition comprising cannabidiol and chamomile blossoms.

### CROSS REFERENCE TO RELATED APPLICATIONS

This application is a national phase application of International Application No.: PCT/EP 2020/082952, filed on Nov. 20, 2020, the content of which is herein incorporated by reference.

### BACKGROUND OF THE INVENTION

### Field of the Invention

The invention relates to a tampon with a base body comprising a pharmaceutical composition and to a method of manufacturing such a tampon.

### Description of Related Art

The use of hemp fibres and/or cannabidiol in tampons for the purpose of reducing menstrual cramps is known from the prior art. For example, WO 2020/026209 A1 (Anne's Day Ltd.) describes a tampon with a base body made from fibres of industrial hemp. In addition, a pharmaceutical composition of cannabidiol and cocoa oil is applied to the surface of the tampon.

### BRIEF SUMMARY OF THE INVENTION

The object of the invention is to provide an improved tampon belonging to the technical field mentioned at the beginning, which in particular provides an especially effective relief from menstrual cramps.

The solution to the object is defined by the features of claim 1. According to the invention, the tampon has an absorbent base body made of cotton comprising a pharmaceutical composition. The pharmaceutical composition comprises cannabidiol and chamomile flowers.

It was surprisingly found that the inventive combination of cannabidiol and chamomile flowers leads to an especially effective and lasting relief of menstrual cramps.

The absorbent base body has a cylindrical base form in a known manner, whereas a distal end has a round or substantially conical tip to facilitate the insertion of the tampon. A proximal end of the tampon has a retrieval string. Preferably, the base body has a plurality of grooves which run either parallel to a longitudinal axis of the base body, or alternatively, wrap partially around the base body in a helical fashion, also known as curved grooves.

In particular, the base body has a length of 40 to 60 mm, especially 45 to 50 mm, with a diameter of 10 mm to 20 mm, preferably 11 to 15 mm.

The cotton is preferably a fleece made of cotton fibres. By using cotton for the base body, the inventive tampon is biodegradable and therefore particularly environmentally friendly.

The cannabidiol is preferably present in the form of an oil in the pharmaceutical composition. The chamomile flowers are preferably the flowers of the true chamomile (Matricaria chamomilla).

The pharmaceutical composition may preferably also comprise further components having a therapeutic effect, for example an extract or a tincture of mallow (Malva Sylvestris), sage (Salvia Officinalis), balm (Melissa Officinalis), yarrow (Achillea Millefolium), lady's mantle (Alchemilla vulgaris), herb of grace (Gratiola Officinalis), purple dead-nettle (Lamium purpureum) or a mixture thereof.

Further, the pharmaceutical composition may preferably comprise at least one additive component that imparts a particular property to the pharmaceutical composition, for example to regulate the release of cannabidiol from the tampon or to adjust the consistency, the pH or the moisture content of the pharmaceutical composition.

The pharmaceutical composition preferably comprises at least one degradable polymer, in particular a natural polymer, for example a polysaccharide such as alginate, dextran, cellulose or collagen. Furthermore, the pharmaceutical composition may also comprise proteins such as, for example, albumin or gelatin.

Further, the pharmaceutical composition may also comprise at least one hydrophobic material, for example wax, a solid fatty acid, hydrogenated vegetable oil, cocoa oil, solid triglycerides, cocoa butter, alkyl silicones or the like.

The pharmaceutical composition is preferably diffused into the base body, wherein the pharmaceutical composition is in particular uniformly distributed in the volume of the base body. Alternatively, however, the pharmaceutical composition may be present with a decreasing concentration from the surface towards the longitudinal axis of the base body.

In an alternatively preferred embodiment, the pharmaceutical composition may also be present only in a partial region of the base body, in particular at the distal end, i.e. at the end that is closer to the cervix when the tampon is in use.

Preferably, the pharmaceutical composition present on the base body comprises 1 to 400 mg, in particular 2 to 250 mg, cannabidiol. At this concentration of cannabidiol per tampon, a particularly good therapeutic effect is achieved in regards to menstrual cramps.

Preferably, the pharmaceutical composition comprises cannabidiol in an amount of 0.1 to 99.9% by weight based on the total weight of the pharmaceutical composition. Especially, the pharmaceutical composition comprises cannabidiol in an amount of 1 to 95% by weight, in particular of 10 to 90% by weight, most preferably of 30 to 80% by weight, based on the total weight of the pharmaceutical composition.

Preferably, the pharmaceutical composition comprises the cannabidiol in a ratio of 1:1 to 25:1, in particular of 5:1 to 10:1, to the chamomile flowers.

Preferably, the chamomile flowers are present in the form of an extract, a tincture or an oil in the pharmaceutical composition. Particularly preferred, the chamomile flowers are present in the form of an aqueous tincture.

Preferably, the cotton is organically cultivated cotton. This can further increase the environmental compatibility of the inventive tampon.

Preferably, the pharmaceutical composition is applied to the surface of the base body. This allows the pharmaceutical composition to pass particularly quickly from the tampon to the mucous membranes of a person who has inserted the tampon.

In an alternatively preferred embodiment, the pharmaceutical composition may be diffused into the base body at a depth of less than 3 mm, in particular less than 1 mm, from the surface of the base body. By diffusing into a zone close to the surface, the active ingredients from the pharmaceutical composition can be rapidly absorbed by the mucous membranes when the tampon is inserted, whereas before the insertion of the tampon the pharmaceutical composition is well protected from external influences.

According to a further advantageous embodiment, the tampon has an application aid and/or is accommodated in an application aid. The application aid serves to support the insertion of the tampon. In particular, the tampon is present inside the application aid.

In particular, the application aid has an elongated outer cylinder, a tampon contained therein and an ejection part with which the tampon can be pushed out of a dispensing opening located at the front end of the outer cylinder. Thereby, the tampon moves out of the outer cylinder along the longitudinal axis of the outer cylinder.

However, other application aids can also be provided.

The present application further relates to a method of manufacturing a tampon. In a first step, a base body of cotton is produced. Subsequently, the base body is treated with a pharmaceutical composition comprising cannabidiol and chamomile flowers. The cotton is preferably organically cultivated. The method according to the invention is used in particular to produce a tampon as described above.

The production of the base body occurs in a known manner by winding a strip of cotton, in particular a cotton fleece. Preferably, during winding, a retrieval string is wrapped in at the proximal end.

The pharmaceutical composition preferably comprises 1 to 400 mg, in particular 2 to 250 mg of cannabidiol.

Preferably, the pharmaceutical composition comprises cannabidiol in an amount of 0.1 to 99.9% by weight based on the total weight of the pharmaceutical composition. Especially, the pharmaceutical composition comprises cannabidiol in an amount of 1 to 95% by weight, in particular of 10 to 90% by weight, most preferably of 30 to 80% by weight, based on the total weight of the pharmaceutical composition.

The pharmaceutical composition is preferably applied to the surface of the base body.

Preferably, the pharmaceutical composition is diffused into the base body to a depth of less than 5 mm, in particular less than 3 mm, from the surface of the base body.

According to a preferred embodiment, the tampon is provided with an application aid and/or is accommodated in an application aid, in particular in an application aid as described above.

Further advantageous embodiments and combinations of features of the invention result from the following detailed description and the totality of the patent claims.

### BRIEF DESCRIPTION OF THE SEVERAL VIEWS OF THE DRAWINGS

Further advantages, features, and details of the various embodiments of this disclosure will become apparent from the ensuring description of a preferred exemplary embodiment and with the aid of the drawings. The features and combinations of features recited below in the description, as well as the features and feature combination shown after that in the drawing description or in the drawings alone, may be used not only in the particular combination received, but also in other combinations on their own, without departing from the scope of the disclosure.

The drawings used to explain the embodiment show:

FIG. 1 a schematic view of a tampon according to the invention.

In principle, the same parts are given the same reference signs in the figures.

### DETAILED DESCRIPTION OF THE INVENTION

As used throughout the present disclosure, unless specifically stated otherwise, the term “or” encompasses all possible combinations, except where infeasible. For example, the expression “A or B” shall mean A alone, B alone, or A and B together. If it is stated that a component includes “A, B or C”, then, unless specifically stated otherwise or infeasible, the component may include A, or B, or C, or A and B, or A and C, or B and C, or A and B and C. Expressions such as “at least one of” do not necessarily modify an entirety of the following list and do not necessarily modify each member of the list, such that at least one of “A, B, and C” should not be understood as including only one of A, only one of B, only one of C, or any combination of A, B, and C.

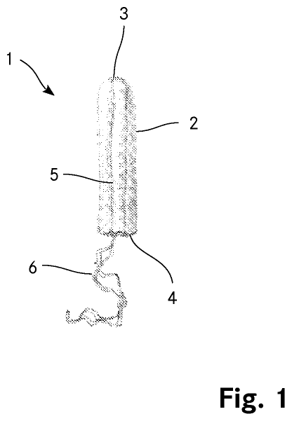

FIG. 1 shows a schematic view of a tampon 1 according to the present invention. The tampon 1 has an absorbent base body 2. In the embodiment shown, the base body 2 is made of a cotton fleece. The base body 2 has a distal end 3 which comes to lie closer to the cervix after insertion of the tampon 1 into the vagina. Furthermore, the base body 2 has a proximal end 4, to which a retrieval string 6 is attached. A plurality of grooves 5 is embedded on the base body 2, which in the shown embodiment are substantially straight, i.e. they run substantially parallel to a longitudinal axis of the base body 2. The base body 2 has a cylindrical base form, whereas the distal end 3 shows a rounding in the form. The base body 2 comprises a pharmaceutical composition comprising cannabidiol and camomile flowers, in particular camomile flower extract.

Since the tampon and its methods of manufacture are described in detail above are examples of embodiments, they can be modified to a wide extent by the skilled person in the usual manner without leaving the scope of the invention. In particular, the arrangements and the proportions of the individual elements with respect to each other are merely exemplary. Some preferred embodiments according to the invention have been disclosed above. The invention is not limited to the solutions explained above, but the innovative solutions can be applied in different ways within the limits set by the claims.

1. A tampon, comprising:
an absorbent base body of comprising cotton and a pharmaceutical composition, and
wherein the pharmaceutical composition comprises cannabidiol and chamomile flowers.

2. The tampon according to claim 1, wherein the pharmaceutical composition comprises 1 to 400 mg of cannabidiol.

3. The tampon according to claim 1, wherein the pharmaceutical composition comprises cannabidiol in an amount of 0.1 to 99.9% by weight based on the total weight of the pharmaceutical composition.

4. The tampon according to claim 1, wherein the chamomile flowers comprise a form of an extract, a tincture or an oil in the pharmaceutical composition.

5. The tampon according to claim 1, wherein the cotton is organically cultivated cotton.

6. The tampon according to claim 1, wherein:
the base body comprises a surface; and
the surface corn rises an application of the pharmaceutical composition thereon.

7. The tampon according to claim 1, wherein the base body comprises:
a surface; and
a diffusion of the pharmaceutical composition into the base body at a depth of less than 3 mm from the surface.

8. The tampon according to claim 1, wherein:
the tampon further comprises an application aid; or
the tampon is configured to be accommodated in the application aid.

9. The tampon according to claim 8, wherein:
the application aid comprises an elongated outer cylinder configured to accommodate the tampon therein and comprising a dispensing opening arranged at a front end of the outer cylinder; and
the application aid further comprises an ejection part arranged and configured to enable the pushing out of which the tampon through the dispensing opening.

10. A method of manufacturing a tampon comprising the steps of:
a) manufacturing a base body of cotton, the base body comprising a surface; and
b) treating the base body with a pharmaceutical composition comprising cannabidiol and chamomile flowers.

11. The method according to claim 10, wherein the pharmaceutical composition comprises) to 400 mg of cannabidiol.

12. The method according to claim 10, wherein the pharmaceutical composition comprises cannabidiol in an amount of 0.1 to 99.9% by weight based on the total weight of the pharmaceutical composition.

13. The method according to claim 10, further comprising the steps of applying the pharmaceutical composition to the surface.

14. The method according to claim 10, further comprising the steps of diffusing the pharmaceutical composition into the base body to a depth of less than 5 mm from the surface.

15. The method according to claim 10, further comprising the steps of:
providing an application aid with the tampon; or
accommodating the tampon in the application aid.

16. The method according to claim 15, wherein:
the application aid comprises an elongated outer cylinder configured to accommodate the tampon therein and comprising a dispensing opening arranged at a front end of the outer cylinder; and
the application said further comprises an ejection part arranged and configured to enable the pushing out of the tampon through the dispensing opening.
